(19) United States
(12) Patent Application Publication
(10) Pub. No.: US 2025/0009452 A1
(43) Pub. Date: Jan. 9, 2025

(54) IMPLANTATION SYSTEM AND METHOD FOR COCHLEAR IMPLANT MICROELECTRODE WITH REMOTE MOTION CENTER CONTROL

(71) Applicant: SHANGHAI NINTH PEOPLE'S HOSPITAL AFFILIATED TO SHANGHAI JIAOTONG UNIVERSITY SCHOOL OF MEDICINE, Shanghai (CN)

(72) Inventors: Hao WU, Shanghai (CN); Huan JIA, Shanghai (CN); Haoyue TAN, Shanghai (CN); Weilong GUO, Shanghai (CN); Lianchao LI, Shanghai (CN); Azhar Aulia Saputra, Shanghai (CN)

(21) Appl. No.: 18/763,880

(22) Filed: Jul. 3, 2024

(30) Foreign Application Priority Data
Jul. 4, 2023 (CN) 202310812130.4

(51) Int. Cl. A61B 34/37; A61B 17/00; A61B 17/29; A61B 34/10; A61B 34/30

(52) U.S. Cl. CPC A61B 34/37; A61B 17/00234; A61B 17/29; A61B 34/10; A61B 2017/00199; A61B 2017/00238; A61B 2017/00398; A61B 2034/107; A61B 2034/302

(57) ABSTRACT

An implantation system for a cochlear implant microelectrode with remote motion center control includes: an electrode implantation device including at least one pair of forceps, and a distal end part of the forceps is used as a remote motion center; a six-degree-of-freedom motion mechanism; an input unit, configured to input a surgical trajectory; and a control unit, configured to calculate, a displacement of the forceps in a first body coordinate system, an offset compensation of each motion execution unit at the front end at a corresponding degree of freedom in the first body coordinate system, and an offset compensation of each motion execution unit at the rear end at a corresponding degree of freedom in a second body coordinate system, and drive, based on the corresponding offset compensation and a preset motion priority, the forceps and the corresponding motion execution unit to act.

### CROSS-REFERENCE TO RELATED APPLICATIONS

The application claims priority to Chinese patent application No. 202310812130.4, filed on Jul. 4, 2023. The disclosure of the aforementioned application is hereby incorporated by reference in its entirety.

### FIELD OF TECHNOLOGY

The present disclosure relates to the field of medical instruments, and specifically to an implantation system and method for a cochlear implant microelectrode with remote motion center control.

### BACKGROUND

As a relatively mature method of hearing reconstruction, cochlear implant surgery has been widely put into use in clinical practice. Electrode implantation is the most critical operation in this surgery, requiring operation as gently as possible to protect a fine structure of an inner ear and/or preserve residual hearing to the maximum extent.

At present, technology for preserving the fine structure of the inner ear during cochlear implant surgery heavily depends on surgeon's experience, physical strength, and other factors. Recent studies have shown that during electrode implantation, involuntary slight hand tremors, due to physiological limitations, can cause local trauma, and even result in damage to a basilar membrane of a cochlea when an electrode is implanted into the cochlea, directly affecting the effectiveness of an artificial auditory system.

Currently, research is being conducted on surgical robots for microelectrode implantation, where the electrode is implanted into the cochlea using forceps disposed on the robot. During the actual surgery, the electrode is held by a distal end part of the forceps, referred to as a remote motion center. A space in which the forceps can move is constrained by a facial structure of a patient, such that the displacement and motion direction of the forceps are necessary to be precisely controlled to avoid damaging the fine structure of the inner ear.

Based on a world coordinate system, a doctor uses an operating handle to output control instructions (including motion parameters of the robot in corresponding directions) to the robot. The robot receives these control instructions based on the world coordinate system but moves according to its body coordinate system. As shown in FIG. 1, a target position of a remote motion center A is in a +X-axis direction of the world coordinate system, and 3 mm away from a current position of the remote motion center A. At this time, forceps 10 have an acute angle with a +X axis, and a +x axis of the forceps in a body coordinate system is perpendicular to the forceps. When the robot receives a control instruction to move 3 mm horizontally in the +X-axis direction, the remote motion center will actually move along a +x-axis direction and cannot reach the target position. Adjusting the control instruction based on doctor's experience also carries the risk that the remote motion center will not reach the target position.

Providing an implantation system and method for a cochlear implant microelectrode that can generate motion parameters in all directions for the robot based on the target position of the remote motion center, ensure that the target position can be reached, and implement the multi-pose motion control on the deep remote motion center within a narrow cavity, is an urgent problem to be solved at present.

### SUMMARY

An objective of the present disclosure is to provide an implantation system and method for a cochlear implant microelectrode with remote motion center control. The system can adjust an offset compensation of a six-degree-of-freedom motion mechanism at each degree of freedom based on inputted target position and target pose of a remote motion center, thereby ensuring that the target position of the remote motion center can be accurately reached, and effectively guaranteeing the safety and surgical effect of cochlear implant microelectrode implantation surgery in combination with a set motion priority at each degree of freedom.

To achieve the above objective, the present disclosure provides an implantation system for a cochlear implant microelectrode with remote motion center control, including:

an electrode implantation device, where the electrode implantation device includes at least one forceps, an electrode to be implanted is clamped by the forceps, and a distal end part of the forceps is used as a remote motion center;
a six-degree-of-freedom motion mechanism, where the six-degree-of-freedom motion mechanism has a front end and a rear end, the front end is connected to the electrode implantation device, each of the front end and the rear end is provided with at least one motion execution unit, a first body coordinate system is established based on the forceps and the front end, and a second body coordinate system is established based on the rear end;
an input unit, configured to input a surgical trajectory, where the surgical trajectory includes multiple pieces of target information of the forceps, and the target information includes, in a world coordinate system, a target position of the remote motion center and a target pose of the forceps; and
a control unit, configured to calculate, based on the current target information and next target information of the forceps, offset compensations of the forceps and the motion execution unit at the front end at a corresponding degree of freedom in the current first body coordinate system and an offset compensation of the motion execution unit at the rear end at a corresponding degree of freedom in the second body coordinate system, and drive, based on each of the offset compensations and a preset motion priority, the forceps and the corresponding motion execution unit to act, thereby implementing automatic implantation of the electrode.

In one embodiment, the input unit includes an operating handle through which a doctor inputs a first driving instruction. The first driving instruction includes a motion direction and a motion amount of the forceps in the world coordinate system; the control unit converts the first driving instruction into a second driving instruction; the second driving instruction includes the offset compensations of the forceps and the motion execution unit at the front end at the corresponding degree of freedom in the first body coordinate system; the second driving instruction further includes the offset compensation of the motion execution unit at the rear end at the corresponding degree of freedom in the second body coordinate system; and based on the second driving instruction and the preset motion priority, the forceps and the corresponding motion execution unit are driven to act, thereby implementing manual implantation of the electrode.

In one embodiment, the front end is provided with a first rotary motion execution unit; the first rotary motion execution unit includes a first driving motor, a first holder, and at least one winding arm;

the first holder is fixedly connected to the first driving motor; the first holder includes two extension arms disposed opposite to each other, and the electrode implantation device is located between the two extension arms and is hinged to the extension arms;
the winding arm has a first end hinged to the electrode implantation device and a second end coupled to a first output shaft of the first driving motor; and the second end of the winding arm is driven by the first driving motor to rotate about the first output shaft, such that the forceps rotate about hinge points of the extension arms and the electrode implantation device.

In one embodiment, the front end is further provided with a second rotary motion execution unit; the second rotary motion execution unit includes a second driving motor; the first driving motor is coupled to a second output shaft of the second driving motor, and the second output shaft is perpendicular to the first output shaft; and the first rotary motion execution unit and the electrode implantation device are driven by the second driving motor to rotate about a central axis of the second output shaft in an integrated manner.

In one embodiment, the rear end is provided with a first rectilinear motion execution unit; the first rectilinear motion execution unit includes a third driving motor, a first gear, a first rectilinear motion mechanism, and a first guide mechanism;

a first end of the first rectilinear motion mechanism is fixedly connected to the second rotary motion execution unit; the first guide mechanism is sleeved on the first rectilinear motion mechanism and is slidably connected to the first rectilinear motion mechanism, and the first rectilinear motion mechanism is guided by the first guide mechanism to move linearly along a Y-axis direction; the Y-axis direction is parallel to a length direction of the second output shaft;
a first gear and guide groove structure extending along the Y-axis direction is formed at the bottom of the first guide mechanism; the third driving motor is fixedly connected to the first rectilinear motion mechanism; the first gear is coupled to an output shaft of the third driving motor and is engaged with the first gear and guide groove structure; and the first gear is driven to rotate, such that the first rectilinear motion mechanism drives the second rotary motion execution unit, the first rotary motion execution unit, and the electrode implantation device to move linearly along the Y-axis direction in an integrated manner.

In one embodiment, the rear end is further provided with a second rectilinear motion execution unit; the second rectilinear motion execution unit includes a second rectilinear motion mechanism, a connecting plate, a fourth driving motor, a fourth gear, and a second guide mechanism;

the connecting plate is fixedly disposed at the top of the first guide mechanism and does not interfere with the first rectilinear motion mechanism; the second rectilinear motion mechanism is fixedly disposed on a top surface of the connecting plate; the second guide mechanism is sleeved on the second rectilinear motion mechanism and is slidably connected to the second rectilinear motion mechanism, and the second rectilinear motion mechanism is guided by the second guide mechanism to move linearly along an X-axis direction; the X-axis direction is perpendicular to the Y-axis direction;
a second gear and guide groove structure extending along a length direction of an X axis is formed on one side of the connecting plate facing away from the electrode implantation device;
the fourth driving motor is fixedly connected to the second guide mechanism; the fourth gear is coupled to an output shaft of the fourth driving motor and is engaged with the second gear and guide groove structure; and the fourth gear is driven to rotate, such that the second rectilinear motion mechanism drives the first rectilinear motion execution unit, the second rotary motion execution unit, the first rotary motion execution unit, and the electrode implantation device to move linearly along the X-axis direction in an integrated manner.

In one embodiment, the rear end is further provided with a third rotary motion execution unit; the third rotary motion execution unit includes a first arc-shaped seat, a second arc-shaped seat, a third rotary motion mechanism, a fifth driving motor, and a fifth gear;

the first arc-shaped seat and the second arc-shaped seat are concentric and have the same radius; the first arc-shaped seat is fixedly disposed at the top of the second arc-shaped seat; an inner arc surface of the first arc-shaped seat faces the electrode implantation device; an outer arc surface of the first arc-shaped seat is provided with an arc-shaped gear and guide groove structure extending along a circumferential direction of the first arc-shaped seat; an inner arc surface and an outer arc surface of the second arc-shaped seat are provided with protrusions protruding outwards, and the protrusions extend along a circumferential direction of the second arc-shaped seat;
the third rotary motion mechanism is disposed below the second arc-shaped seat, and a top surface of the third rotary motion mechanism is fixedly provided with a plurality of upright columns, and the plurality of upright columns are located on an inner side and an outer side of the second arc-shaped seat respectively; outer walls of the upright columns are sunken inwards to form recesses, and the protrusions are partially embedded into the recesses; the protrusions are matched with the recesses, such that the second arc-shaped seat guides the third rotary motion mechanism to rotate about a Z axis; the Z axis is a central axis of the second arc-shaped seat that is perpendicular to the X axis and a Y axis;
the third rotary motion mechanism is further fixedly connected to the second guide mechanism and the fifth driving motor; the fifth gear is coupled to an output shaft of the fifth driving motor and is engaged with the arc-shaped gear and guide groove structure; and the fifth gear is driven to rotate, such that the third rotary motion mechanism drives the second rectilinear motion execution unit, the first rectilinear motion execution unit, the second rotary motion execution unit, the first rotary motion execution unit, and the electrode implantation device to rotate about the Z axis in an integrated manner.

In one embodiment, the rear end is further provided with a third rectilinear motion execution unit, and the third rectilinear motion execution unit includes a third guide mechanism, a sixth driving motor, a sixth gear, and a third rectilinear motion mechanism;

the third guide mechanism is fixedly connected to an external mechanism and the sixth driving motor; the third guide mechanism is sleeved on the third rectilinear motion mechanism and is slidably connected to the third rectilinear motion mechanism; the third rectilinear motion mechanism is guided by the third guide mechanism to move linearly along a direction parallel to the Z axis;
the third rectilinear motion mechanism is fixedly connected to the second arc-shaped seat; the third rectilinear motion mechanism is further provided with a third gear and guide groove structure extending along a Z-axis direction;
the sixth gear is coupled to an output shaft of the sixth driving motor and is engaged with the third gear and guide groove structure; and the sixth gear is driven by the sixth driving motor to rotate, such that the third rectilinear motion mechanism drives the third rotary motion execution unit, the second rectilinear motion execution unit, the first rectilinear motion execution unit, the second rotary motion execution unit, the first rotary motion execution unit, and the electrode implantation device to move linearly along the Z-axis direction in an integrated manner.

In one embodiment, the electrode implantation device further includes at least one first implantation motor, and one of the first implantation motors is corresponding to one pair of forceps; the forceps are driven by the first implantation motor to move linearly along a direction perpendicular to the first output shaft;

the first rotary motion execution unit and the second rotary motion execution unit have the same motion priority;
the first rectilinear motion execution unit and the second rectilinear motion execution unit have the same motion priority; and
motion priorities of the first implantation motor, the first rotary motion execution unit, the first rectilinear motion execution unit, the third rotary motion execution unit, and the third rectilinear motion execution unit decrease progressively in sequence.

The present disclosure further provides an implantation method for a cochlear implant microelectrode, implemented with the implantation system for a cochlear implant microelectrode as described in the present disclosure, and including the following operations:
S1: in an automatic implantation mode, entering S2; or in a manual implantation mode, entering S4;
S2: inputting a surgical trajectory, where the surgical trajectory includes multiple pieces of target information of a pair of forceps;
S3: calculating, based on current target information and next target information of the forceps, offset compensations of the forceps and a motion execution unit at a front end at a corresponding degree of freedom in a first body coordinate system and an offset compensation of a motion execution unit at a rear end at a corresponding degree of freedom in a second body coordinate system; and driving, based on each of the offset compensations and a preset motion priority, the forceps and the corresponding motion execution unit to act, thereby implementing automatic implantation of an electrode;
S4: inputting a first driving instruction by an operating handle, where the first driving instruction includes a motion direction and a motion amount of the forceps in a world coordinate system; and
S5: converting, by a control unit, the first driving instruction into a second driving instruction, where the second driving instruction includes the offset compensations of the forceps and the motion execution unit at the front end at the corresponding degree of freedom in the first body coordinate system, and the second driving instruction further includes the offset compensation of the motion execution unit at the rear end at the corresponding degree of freedom in the second body coordinate system; and driving, based on the second driving instruction and the preset motion priority, the forceps and the corresponding motion execution unit to act, thereby implementing manual implantation of the electrode.

Compared with the prior art, the present disclosure has the following beneficial effects:
(1) According to the implantation system and method for a cochlear implant microelectrode with remote motion center control in the present disclosure, a robot technology is used to replace conventional manual implantation, such that the stability during microelectrode implantation is greatly improved, and the risk of damage to fine and delicate structures such as local neurons during electrode implantation is lowered. The present disclosure has high degree of freedom of operation and may be applicable to the conditions of narrow cavity and small surgical field.
(2) The implantation system in the present disclosure has an automatic implantation mode and a manual implantation mode. In the automatic implantation mode, the first body coordinate system corresponding to the front end and the second body coordinate system corresponding to the rear end of the six-degree-of-freedom motion mechanism can be established based on the current target information and the next target information of the forceps in the preset surgical trajectory and based on inverse kinematics, and the offset compensations (linear distance and direction of motion or angle and direction of rotation) at each degree of freedom in the corresponding body coordinate system are automatically generated for the forceps and each motion execution unit, such that the target position in the next target information can be reached, the forceps are in the target pose, the damage to the fine structure of the inner ear is avoided, and the advantages of optimal implantation angle, accurate trajectory, and minimal trauma are achieved. In the manual implantation mode, the control unit can convert the first driving instruction based on the world coordinate system into the second driving instruction, where the second driving instruction includes the offset compensations of the forceps and the corresponding motion execution unit at the corresponding degree of freedom in the first body coordinate system or the second body coordinate system, which ensures that the motion of the deep remote motion center within the narrow cavity meets the expectation of a doctor, thereby ensuring the safety of complex surgery requiring manual intervention.
(3) The six motion execution units of the six-degree-of-freedom motion mechanism are all decoupled, such that the combination of respective independent motion and required motion can be implemented, thereby meeting the surgical requirements. The electrode implantation device and the six-degree-of-freedom motion mechanism in the present disclosure are small in size, such that the bearing requirements of the rear end can be reduced.
(4) The electrode implantation device in the present disclosure may be a dual-forceps design including two sets of clamping components, and may be well compatible with the implantation of an artificial auditory electrode with a support structure such as a guide core. During electrode implantation, the forceps may move back and forth for multiple times to push the electrode into the cochlea, such that the size of the electrode implantation device can be effectively reduced, thereby achieving a lightweight design.

### BRIEF DESCRIPTION OF THE DRAWINGS

To more clearly illustrate the technical solutions of the present disclosure, the accompanying drawings that need to be used in the description will be briefly described below. Apparently, the accompanying drawings in the description below merely illustrate one embodiment of the present disclosure. Those of ordinary skill in the art may also derive other accompanying drawings from these accompanying drawings without creative efforts.

FIG. 1 is a schematic diagram in which a control instruction of a doctor is mismatched with motion of a pair of forceps in the prior art;

FIG. 2 is a schematic diagram of an implantation system for a cochlear implant microelectrode with a remote motion center control according to an embodiment of the present disclosure;

FIG. 3 is a three-dimensional view of an electrode implantation device and a six-degree-of-freedom motion mechanism according to an embodiment of the present

FIG. 4 is a schematic exploded view of an electrode implantation device and a six-degree-of-freedom motion mechanism according to an embodiment of the present disclosure;

FIG. 5A is a schematic diagram of an electrode implantation device according to an embodiment of the present disclosure;

FIG. 5B is a schematic diagram of a narrow cavity where a pair of forceps is located during electrode implantation;

FIG. 6 is a schematic diagram of a first rotary motion execution unit according to an embodiment of the present disclosure;

FIG. 7 is a schematic diagram of a second rotary motion execution unit according to an embodiment of the present disclosure;

FIG. 8 is a schematic diagram of a first rectilinear motion execution unit according to an embodiment of the present disclosure;

FIG. 9 is a schematic diagram of a second rectilinear motion execution unit according to an embodiment of the present disclosure;

FIG. TOA is a schematic diagram of a third rotary motion execution unit according to an embodiment of the present disclosure;

FIG. 10B is a schematic diagram in which an upright column of a third rotary motion execution unit is slidably connected to a second arc-shaped section according to an embodiment of the present disclosure;

FIG. 10C is a top view of FIG. 10B;

FIG. 10D is a view of E-E in FIG. 10C;

FIG. 11 is a schematic diagram of a third rectilinear motion execution unit according to an embodiment of the present disclosure; and

FIG. 12 is a flowchart of an implantation method for a cochlear implant microelectrode according to an embodiment of the present disclosure.

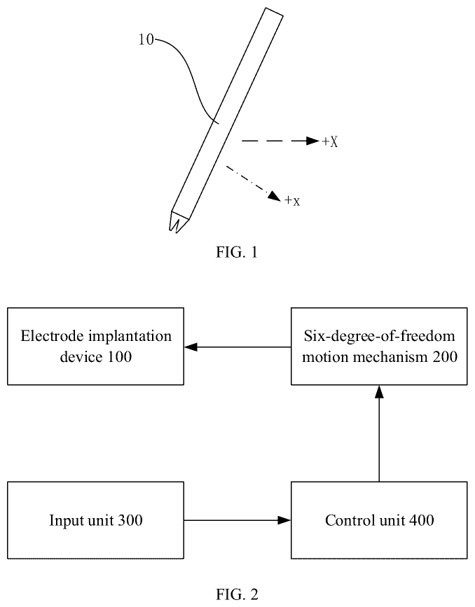

### DETAILED DESCRIPTION OF THE EMBODIMENTS

The technical solutions in the embodiments of the present disclosure will be clearly and completely described below with reference to the accompanying drawings in the embodiments of the present disclosure. Apparently, the described embodiments are merely some rather than all of the embodiments of the present disclosure. All other embodiments obtained by those of ordinary skill in the art based on the embodiments in the present disclosure without creative efforts shall fall within the scope of protection of the present disclosure.

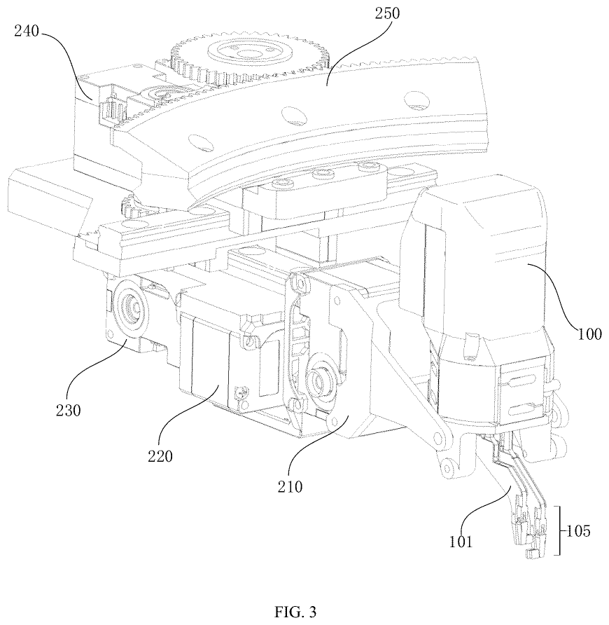

The present disclosure provides an implantation system for a cochlear implant microelectrode with remote motion center control, as shown in FIG. 2, including an electrode implantation device 100, a six-degree-of-freedom motion mechanism 200, an input unit 300, and a control unit 400.

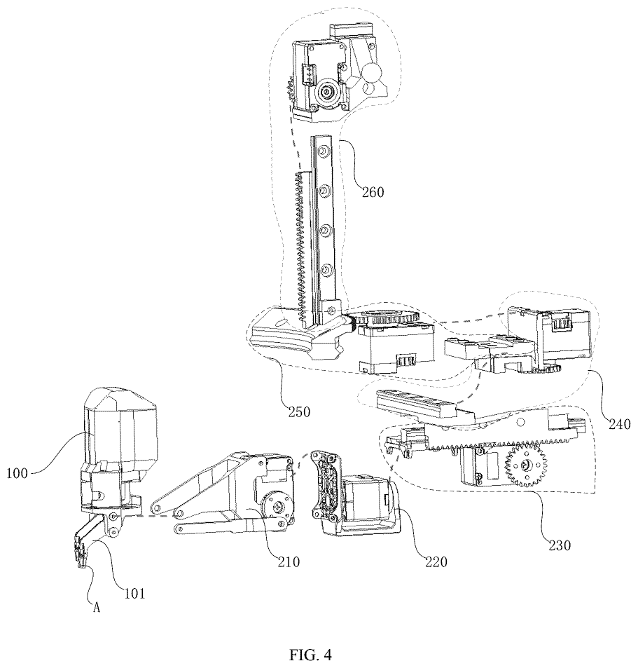

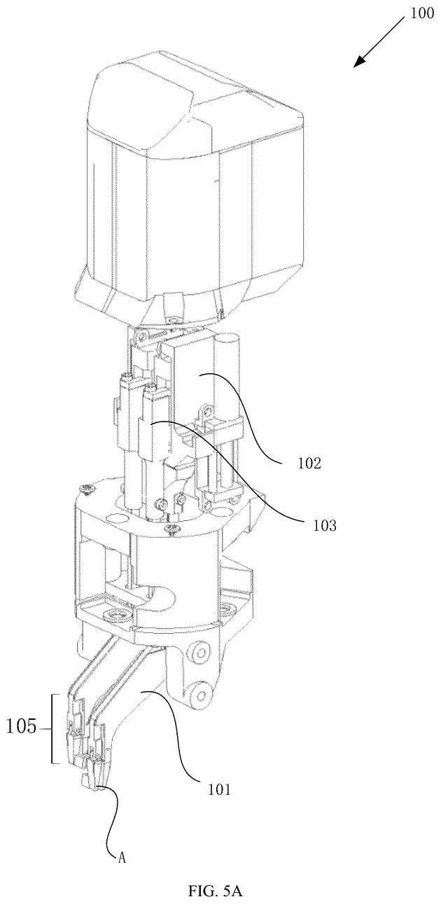

FIG. 3 is a three-dimensional view of the electrode implantation device 100 and the six-degree-of-freedom motion mechanism 200 in the present embodiment. FIG. 4 is a schematic exploded view of the electrode implantation device 100 and the six-degree-of-freedom motion mechanism 200 in the present embodiment.

As shown in FIG. 3 to FIG. 5A, the electrode implantation device 100 includes at least one pair of forceps 101, an electrode to be implanted is clamped by the forceps 101, and a distal end part of the forceps is used as a remote motion center A. As shown in FIG. 3 to FIG. 5A, the forceps 101 in the present embodiment is of a bent structure, such that the surgical field can be prevented from being blocked as much as possible.

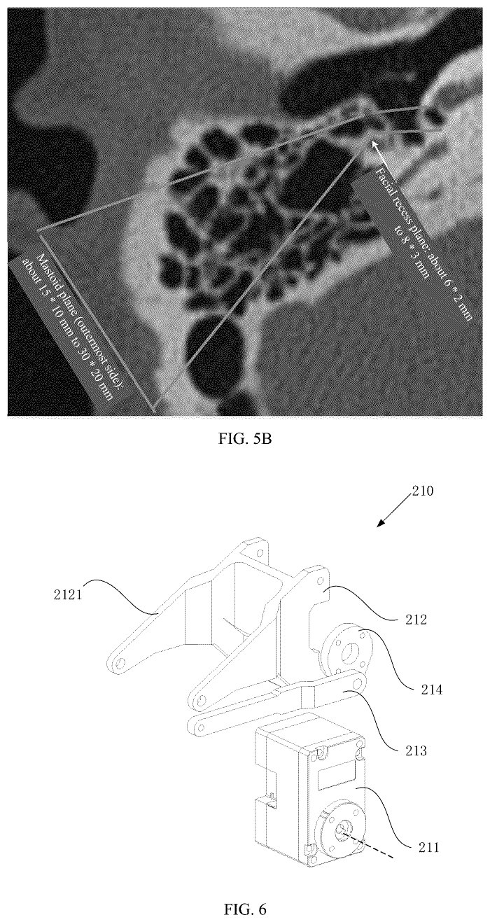

In the present embodiment, the number of the forceps 101 is two pairs, and the electrode implantation device 100 further has two first implantation motors 102 corresponding to the two pairs of forceps 101 respectively. As shown in FIG. 3 and FIG. 5A, the forceps have a distal end section 105 in a straight line shape. The forceps 101 are driven by the first implantation motor 102 to move along a length direction of the distal end section 105. FIG. 5A further shows two second implantation motors 103 configured to drive the opening and closing of two pairs of forceps 101 respectively. The forceps 101 may move back and forth for multiple times to push the electrode into a cochlea, such that the size of the electrode implantation device 100 can be effectively reduced, thereby achieving a lightweight design. In the present disclosure, the two forceps 101 may be well compatible with the implantation of the electrode with a support structure such as a guide core. During electrode implantation, the forceps 101 may move back and forth for multiple times to push the electrode into the cochlea, such that the size of the electrode implantation device 100 can be effectively reduced, thereby achieving a lightweight design.

During the actual surgery, a space in which the forceps can move is constrained by a facial structure of a patient and a designed activity space. As shown in FIG. 5B, a main body of the forceps 101 moves within an inverted trapezoidal range from a mastoid plane (an outermost side) to a facial recess plane (the depth of about 20 mm), where a sectional area of the mastoid plane, namely the outermost side is about 15 mm*10 mm to 30 mm*20 mm. A tip of the forceps 101 moves within a rectangular range from the facial recess plane to a round fenestra (the depth of about 5 mm), where a sectional area of the facial recess plane is about 6 mm*2 mm to 8 mm*3 mm. It can be clearly seen that the surgery in the present disclosure is performed within a very narrow cavity.

In the present disclosure, the six-degree-of-freedom motion mechanism 200 has a front end and a rear end. As shown in FIG. 4, the six-degree-of-freedom motion mechanism 200 has a front end and a rear end. The front end is provided with a first rotary motion execution unit 210 and a second rotary motion execution unit 220. The rear end is provided with a first rectilinear motion execution unit 230, a second rectilinear motion execution unit 240, a third rotary motion execution unit 250, and a third rectilinear motion execution unit 260.

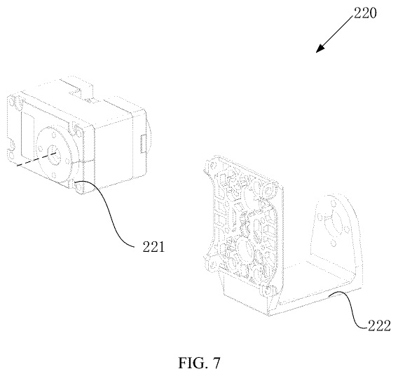

As shown in FIG. 6, the first rotary motion execution unit 210 includes a first driving motor 211, a first holder 212, and at least one winding arm 213. In the present embodiment, the first rotary motion execution unit 210 includes two winding arms 213.

The first holder 212 is fixedly connected to the first driving motor 211, and the first holder 212 includes two extension arms 2121 disposed opposite to each other. The electrode implantation device 100 is located between the two extension arms 2121 and is hinged to the two extension arms 2121.

A first end of the winding arm 213 is hinged to the electrode implantation device 100. A second end of the winding arm 213 is coupled to a first output shaft (which is not shown in the figure, and a dashed line in FIG. 6 represents a central axis of the first output shaft) of the first driving motor 211 by a coupling 214. The coupling 214 is driven by the first driving motor 211 to rotate about the central axis of the first output shaft, such that the second end of the winding arm 213 rotates about the first output shaft, the forceps 101 rotate about hinge points of the extension arms 2121 and the electrode implantation device 100, and the electrode implantation device 100 makes a pitch motion.

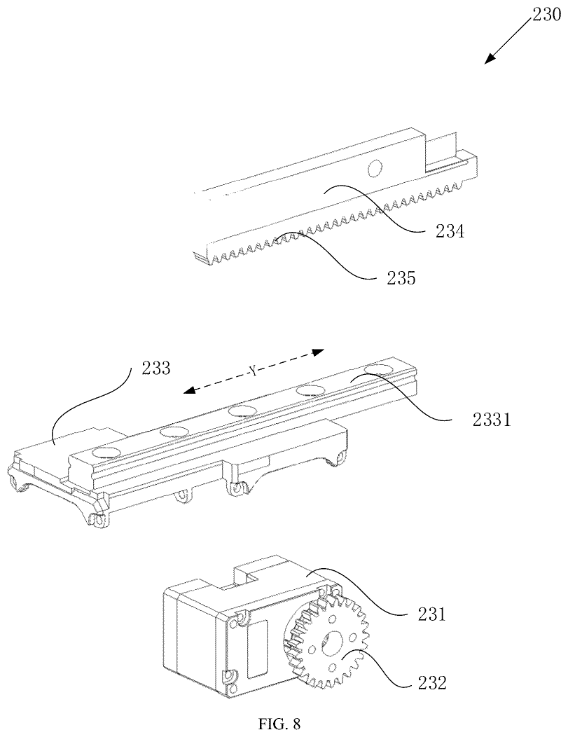

As shown in FIG. 7, the second rotary motion execution unit 220 includes a second driving motor 221 fixedly disposed on a motor base 222. The first driving motor 211 is coupled to a second output shaft (which is not shown in the figure, and a dashed line in FIG. 7 represents a central axis of the second output shaft) of the second driving motor 221, and the second output shaft is perpendicular to the first output shaft. The first rotary motion execution unit 210 and the motor implantation device 100 are driven by the second driving motor 221 to rotate about the central axis of the second output shaft in an integrated manner.

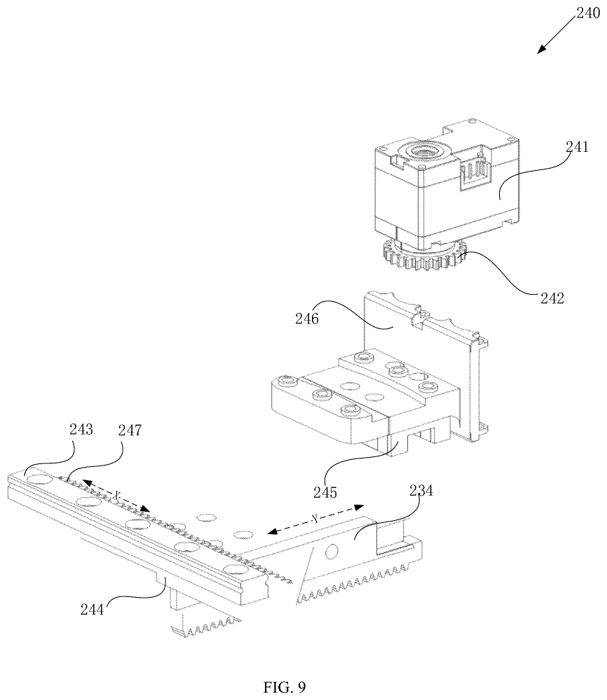

As shown in FIG. 8, the first rectilinear motion execution unit 230 includes a third driving motor 231, a first gear 232, a first rectilinear motion mechanism 233, and a first guide mechanism 234.

In the present embodiment, the first rectilinear motion mechanism 233 includes a rail-like structure 2331 in a straight line shape, where a length direction of the rail-like structure 2331 is parallel to the second output shaft. A first end of the first rectilinear motion mechanism 233 is fixedly connected to the motor base 222 of the second rotary motion execution unit 220. As shown in FIG. 4 and FIG. 8, a U-shaped groove structure is formed at the bottom of the first guide mechanism 234, and the U-shaped groove structure is sleeved on the rail-like structure 2331 and is slidably connected to the rail-like structure 2331. The first rectilinear motion mechanism 233 is guided by the first guide mechanism 234 to move linearly along a Y-axis direction. The Y-axis direction is the length direction of the rail-like structure 2331, which is parallel to a length direction of the second output shaft.

As shown in FIG. 8, a first gear and guide groove structure 235 extending along the Y-axis direction is formed at the bottom of the first guide mechanism 234. The third driving motor 231 is fixedly disposed at the bottom of the first rectilinear motion mechanism 233, and the first gear 231 is coupled to an output shaft of the third driving motor 231 and is engaged with the first gear and guide groove structure 235. The first gear 231 is driven to rotate, such that the first rectilinear motion mechanism 233 drives the second rotary motion execution unit 220, the first rotary motion execution unit 210, and the electrode implantation device 100 to move linearly along the Y-axis direction in an integrated manner.

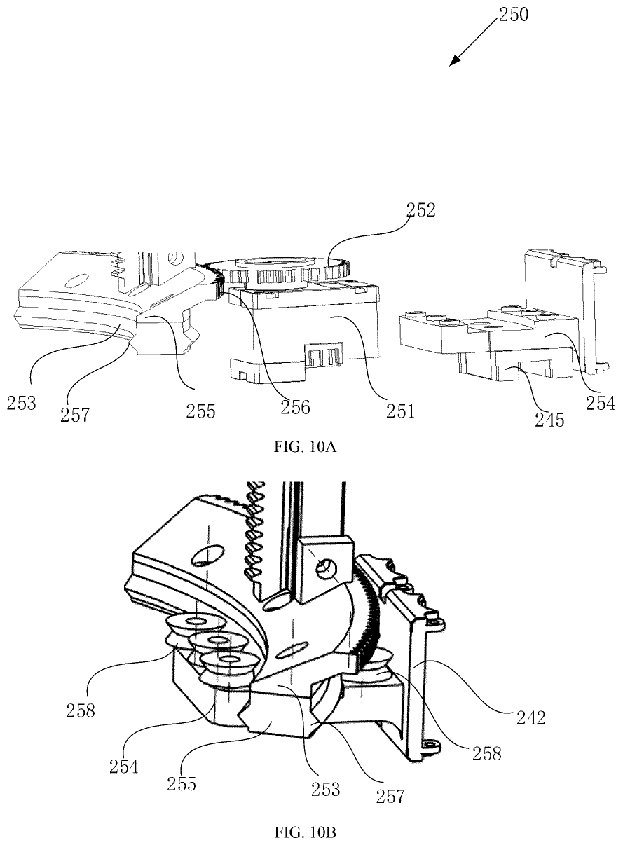

As shown in FIG. 9, the second rectilinear motion execution unit 240 includes a second rectilinear motion mechanism 243, a connecting plate 244, a fourth driving motor 241, a fourth gear 242, and a second guide mechanism 245.

As shown in FIG. 9, the connecting plate 244 is fixedly disposed at the top of the first guide mechanism 234 and does not interfere with the first rectilinear motion mechanism 230. A second gear and guide groove structure 247 extending along an X-axis direction is formed on one side of the connecting plate 244 facing away from the electrode implantation device. As shown in FIG. 9, the X-axis direction is perpendicular to the Y-axis direction.

The second rectilinear motion mechanism 243 has a track structure in a straight line shape fixedly disposed on a top surface of the connecting plate 244. A length direction of the second rectilinear motion mechanism 243 is parallel to the X-axis direction. In the present embodiment, the second guide mechanism 245 has a U-shaped groove structure which is sleeved on the second rectilinear motion mechanism 243 and is slidably connected to the second rectilinear motion mechanism 243. The second rectilinear motion mechanism 243 is guided by the second guide mechanism 245 to move linearly along the X-axis direction.

As shown in FIG. 9, the fourth driving motor 241 is fixedly connected to the second guide mechanism 245 by a mounting plate 246, and the fourth driving motor 241 and the second guide mechanism 245 are located on two sides of the mounting plate 246 respectively. The fourth gear 242 is coupled to an output shaft of the fourth driving motor 241 and is engaged with the second gear and guide groove structure 247. The fourth gear 242 is driven to rotate, such that the second rectilinear motion mechanism 243 drives the first rectilinear motion execution unit 230, the second rotary motion execution unit 220, the first rotary motion execution unit 210, and the electrode implantation device 100 to move linearly along the X-axis direction in an integrated manner.

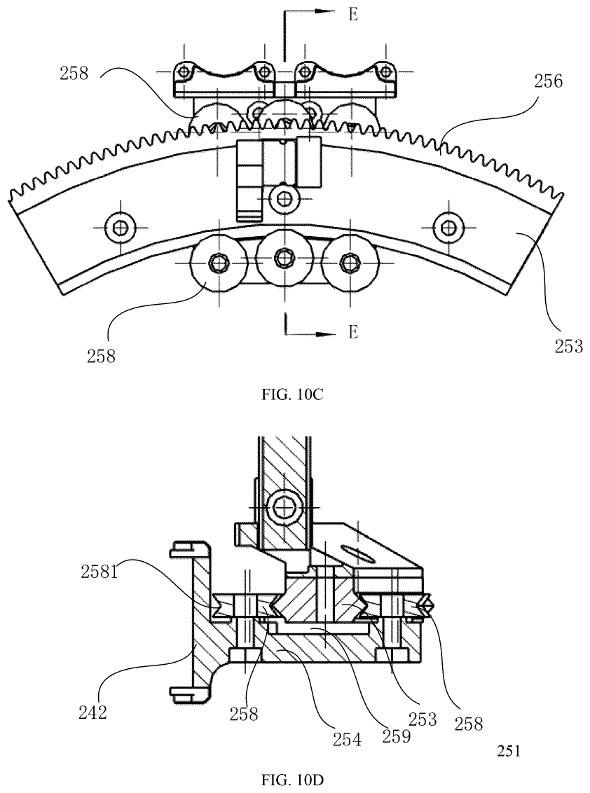

As shown in FIG. 10A, the third rotary motion execution unit 250 includes a first arc-shaped seat 255, a second arc-shaped seat 253, a third rotary motion mechanism 254, a fifth driving motor 251, and a fifth gear 252.

As shown in FIG. 10A, the first arc-shaped seat 255 and the second arc-shaped seat 253 are concentric and have the same radius, the first arc-shaped seat 255 is fixedly disposed at the top of the second arc-shaped seat 253, and an inner arc surface of the first arc-shaped seat 255 faces the electrode implantation device. An outer arc surface of the first arc-shaped seat 255 is provided with an arc-shaped gear and guide groove structure 256 extending along a circumferential direction of the first arc-shaped seat 255. An inner arc surface and an outer arc surface of the second arc-shaped seat 253 are provided with protrusions 257 protruding outwards, and the protrusions 257 extend along a circumferential direction of the second arc-shaped seat 253.

The third rotary motion mechanism 254 is located below the second arc-shaped seat 253, and a bottom surface of the third rotary motion mechanism 254 is fixedly connected to a top surface of the second guide mechanism 245. As shown in FIG. 10B, a top surface of the third rotary motion mechanism 254 is fixedly provided with a plurality of upright columns 258, and the plurality of upright columns 258 are located on an inner side and an outer side of the second arc-shaped seat 253 respectively. FIG. 10C is a top view of FIG. 10B. Three upright columns 258 (the number of the upright columns 258 is only used as an example and not as a limitation to the present disclosure) are fixedly disposed on each of the inner side and the outer side of the second arc-shaped seat 253. Projections of central points of the three upright columns 258 on the inner side and central points of the three upright columns 258 on the outer side on the top surface of the third rotary motion mechanism 254 are located on a first virtual arc line and a second virtual arc line respectively, and the first arc line and the second arc line are coaxial with the second arc-shaped seat 253. FIG. 10D is a view of E-E in FIG. 10C. As shown in FIG. 10D, the top surface of the third rotary motion mechanism 254 is further provided with an arc-shaped groove 259, thereby preventing the third rotary motion mechanism 254 from interfering with the second arc-shaped seat 253.

As shown in FIG. 10D, outer walls of the upright columns 258 are sunken inwards to form recesses 2581. The protrusions 257 of the inner arc surface and the outer arc surface of the second arc-shaped seat 253 are partially embedded into the recesses 2581 of the upright columns 258 on the inner side and the outer side of the second arc-shaped seat 253. The protrusions 257 are matched with the recesses 2581, such that the second arc-shaped seat 253 guides the third rotary motion mechanism 254 to rotate about a Z axis. The Z axis is a central axis of the second arc-shaped seat 253 that is perpendicular to an X axis and a Y axis.

The third rotary motion mechanism 254 is further fixedly connected to the second guide mechanism 245 and the connecting plate 244. The protrusions 257 are matched with the recesses 2581, such that the second rectilinear motion execution unit 240 is hung on the second arc-shaped seat 253. The fifth gear 252 is coupled to an output shaft of the fifth driving motor 251 and is engaged with the arc-shaped gear and guide groove structure 256. The fifth gear 252 is driven to rotate, such that the third rotary motion mechanism 254 drives the second rectilinear motion execution unit 240, the first rectilinear motion execution unit 230, the second rotary motion execution unit 220, the first rotary motion execution unit 210, and the electrode implantation device 100 to rotate about the Z axis in an integrated manner.

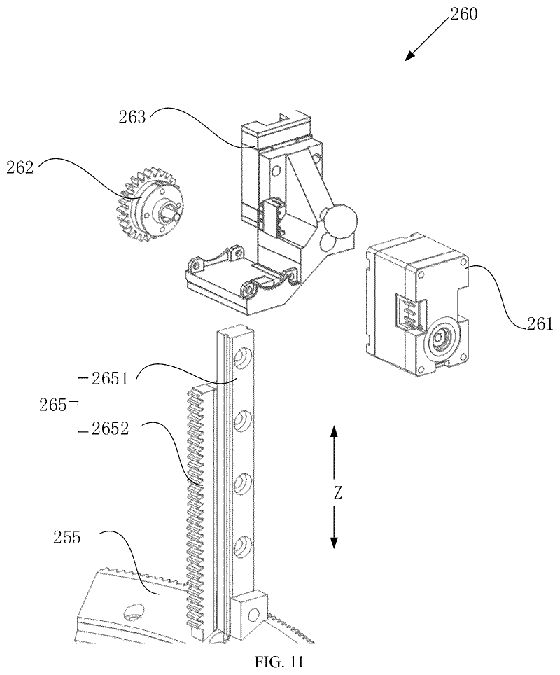

As shown in FIG. 11, the third rectilinear motion execution unit 260 includes a third guide mechanism 263, a sixth driving motor 261, a sixth gear 262, and a third rectilinear motion mechanism 265.

The third rectilinear motion mechanism 265 is fixedly connected to the second arc-shaped seat 253, and the third rectilinear motion mechanism 265 is further provided with a third gear and guide groove structure 2652 extending along a Z-axis direction, and a rail-like structure 2651.

The third guide mechanism 263 is fixedly connected to an external mechanism (such as a robotic arm or the patient's skull) and the sixth driving motor 261. As shown in FIG. 11, the third guide mechanism 263 has a U-shaped groove structure, and the U-shaped groove structure is sleeved on the rail-like structure 2651 and is slidably connected to the rail-like structure 2651. The third rectilinear motion mechanism 265 is guided by the third guide mechanism 263 to move linearly along a direction parallel to the Z axis.

The sixth gear 262 is coupled to an output shaft of the sixth driving motor 261 and is engaged with the third gear and guide groove structure 2652. The sixth gear 262 is driven by the sixth driving motor 261 to rotate, such that the third rectilinear motion mechanism 265 drives the third rotary motion execution unit 250, the second rectilinear motion execution unit 240, the first rectilinear motion execution unit 230, the second rotary motion execution unit 220, the first rotary motion execution unit 210, and the electrode implantation device 100 to move linearly along the Z-axis direction in an integrated manner.

A first body coordinate system is established based on the forceps 101 and the front end, and a second body coordinate system is established based on the rear end. In the present embodiment, an x axis, a y axis, and a z axis of the first body coordinate system are parallel to the length direction of the distal end section 105 of the forceps, the length direction of the output shaft of the first driving motor, and the length direction of the output shaft of the second driving motor, respectively. In a preferred embodiment, an origin of the first body coordinate system is located on the first output shaft and between the two pairs of forceps 101. In the present embodiment, a Z″ axis of the second body coordinate system is the above Z axis, X″ and Y″ axes of the second body coordinate system are parallel to the above X and Y axes respectively, and an origin of the second body coordinate system is a point (its position remains fixed) on the Z″ axis.

In order to simplify the operation, in a preferred embodiment, a connection point (which is a fixed point in surgery) between the third guide mechanism and the external mechanism is used as an origin of a world coordinate system. A Z′ axis of the world coordinate system is perpendicular to a horizontal plane. In the prior art, a correspondence among the first body coordinate system, the second body coordinate system, and the world coordinate system may be acquired in real time through an image containing the forceps 101 and the six-degree-of-freedom motion mechanism that is captured by a camera (this is the prior art and will not be repeated herein).

Due to the fact that the forceps 101 will be driven by the first rotary motion execution unit 210 and the second rotary motion execution unit 110 to rotate, it is readily understood that the first body coordinate system is not equivalent to the second body coordinate system. Under the drive of the first driving motor 211 to the sixth driving motor 261 and the first implantation motor 102, the first body coordinate system and the second body coordinate system are transformed in real time.

In one embodiment, the input unit 300 may be an operating lever. In a manual implantation mode, a doctor inputs a first driving instruction by the operating lever, where the first driving instruction includes a motion direction and a motion amount of the forceps 101 in the world coordinate system. The control unit 400 converts the first driving instruction into a second driving instruction, where the second driving instruction includes offset compensations (direction and displacement of rectilinear motion or direction and angle of rotation) of the forceps 101 and each motion execution unit at the front end at a corresponding degree of freedom in the first body coordinate system; and the second driving instruction further includes an offset compensation of each motion execution unit at the rear end at a corresponding degree of freedom in the second body coordinate system. It is readily understood that the control unit 400 is in signal connection with each driving motor and each implantation motor, and each driving motor and each implantation motor act based on the second driving instruction and preset motion priorities, thereby implementing manual implantation of the electrode.

In another embodiment, the input unit 300 may also be a computer terminal, and a surgical trajectory is inputted to the control unit 400 by the input unit 300, where the surgical trajectory includes multiple pieces of target information of the forceps. The target information includes, in the world coordinate system, a target position of the remote motion center and a target pose of the forceps. The pose of the forceps 101 refers to a pose of the distal end section 105 of the forceps.

The control unit 400 calculates, based on current target information and next target information of the forceps 101, the offset compensations of the forceps and each motion execution unit at the front end at the corresponding degree of freedom in the current first body coordinate system, including the displacement of the forceps 101 on the z axis and the rotation amount (including the direction and angle of rotation) of the forceps 101 rotating about the x axis and the y axis, and the offset compensation of each motion execution unit at the rear end at the corresponding degree of freedom in the second body coordinate system, including the displacements (including the direction and amount of displacement) on the X″, Y″ and Z″ axes and the amount of rotation about the Z″ axis. Based on each of the offset compensations and a preset motion priority, the forceps 101 and the corresponding motion execution unit are driven to act, and when the remote motion center reaches the target position and the forceps 101 reaches the target pose, the first body coordinate system and the second body coordinate system are updated. The above operations are repeated until the surgical trajectory is completed.

In another embodiment, after the third guide mechanism 263 is connected to the external mechanism and before automatic implantation is performed, the doctor may adjust the forceps 101 and the six-degree-of-freedom motion mechanism 200 for the first time based on experience, such that the forceps 101 are in an optimal preoperative attitude. At this time, the remote motion center is usually located near the mastoid plane and has not yet penetrated into the above narrow cavity. The first body coordinate system and the second body coordinate system at this time are used as an initial first body coordinate system and an initial second body coordinate system.

In order to ensure the surgical safety in the narrow cavity, a displacement range and a rotation angle range at each degree of freedom may also be inputted to the control unit 400. When the displacement and the rotation angle in the offset compensation exceed preset displacement range and rotation angle range, the control unit 400 drives an alarm device (not shown in the figure) to give an alarm, and the forceps and each motion execution unit do not act, further avoiding safety accidents caused by misoperation.

In the present embodiment, the first rotary motion execution unit 210 and the second rotary motion execution unit 220 have the same motion priority. The first rectilinear motion execution unit 230 and the second rectilinear motion execution unit 240 have the same motion priority. Motion priorities of the first implantation motor 100, the first rotary motion execution unit 210, the first rectilinear motion execution unit 230, the third rotary motion execution unit 250, and the third rectilinear motion execution unit 260 decrease progressively in sequence. The motion execution units are driven according to the priorities, which can meet the requirements of cochlear implant microelectrode implantation surgery and avoid interference in the narrow cavity.

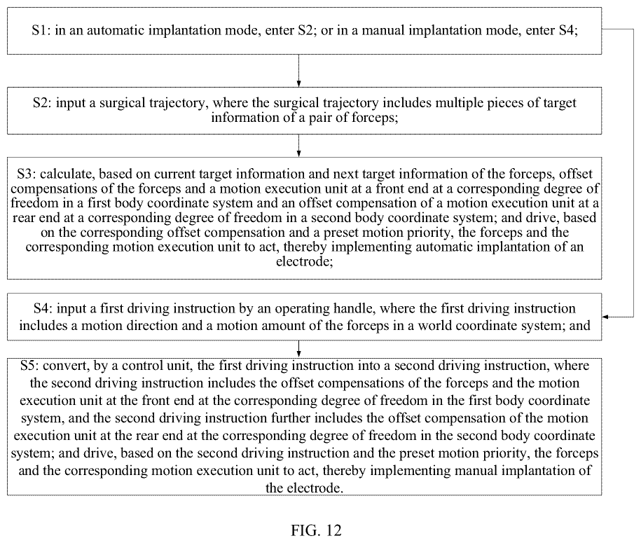

The present disclosure further provides an implantation method for a cochlear implant microelectrode, implemented with the implantation system for a cochlear implant microelectrode as described in the present disclosure, and as shown in FIG. 12, including the following operations:
S1: in an automatic implantation mode, entering S2; or in a manual implantation mode, entering S4;
S2: inputting a surgical trajectory, where the surgical trajectory includes multiple pieces of target information of a pair of forceps;
S3: calculating, based on current target information and next target information of the forceps, offset compensations of the forceps and each motion execution unit at a front end at a corresponding degree of freedom in a first body coordinate system and an offset compensation of each motion execution unit at a rear end at a corresponding degree of freedom in a second body coordinate system; and driving, based on each of the offset compensations and a preset motion priority, the forceps and the corresponding motion execution unit to act, thereby implementing automatic implantation of an electrode;
S4: inputting a first driving instruction by an operating handle, where the first driving instruction includes a motion direction and a motion amount of the forceps in a world coordinate system; and
S5: converting, by a control unit, the first driving instruction into a second driving instruction, where the second driving instruction includes the offset compensations of the forceps and each motion execution unit at the front end at the corresponding degree of freedom in the first body coordinate system, and the second driving instruction further includes the offset compensation of each motion execution unit at the rear end at the corresponding degree of freedom in the second body coordinate system; and driving, based on the second driving instruction and the preset motion priority, the forceps and the corresponding motion execution unit to act, thereby implementing manual implantation of the electrode.

It is to be understood that in the above embodiments, the sequence number of each operation does not mean an execution sequence, and the execution sequence of each process should be determined based on its function and internal logic and should not constitute any limitation to the implementation process of the embodiments of the present application.

The above description is only specific embodiments of the present disclosure, but the scope of protection of the present disclosure is not limited thereto. Any of those skilled in the art may easily think of various equivalent modifications or substitutions within the technical scope of the present disclosure, and these modifications or substitutions should all be included within the scope of protection of the present disclosure. Therefore, the scope of protection of the present disclosure shall be subject to the scope of protection of the claims.

1. An implantation system, for a cochlear implant microelectrode with a remote motion center control, comprising:
an electrode implantation device comprising at least one pair of forceps, wherein an electrode to be implanted is clamped by the at least one pair of forceps, and wherein a distal end part of the at least one pair of forceps is used as a remote motion center;
a six-degree-of-freedom motion mechanism having a front end and a rear end, wherein the front end is connected to the electrode implantation device, each of the front end and the rear end comprises at least one motion execution unit, a first body coordinate system is established based on the at least one pair of forceps and the front end, and a second body coordinate system is established based on the rear end;
an input unit; configured to input a surgical trajectory comprising multiple pieces of target information of the at least one pair of forceps, wherein the target information comprises, in a world coordinate system, a target position of the remote motion center and a target pose of the at least one pair of forceps; and
a control unit configured to calculate, based on current target information and next target information of the at least one pair of forceps, first offset compensations of the at least one pair of forceps and the at least one motion execution unit at the front end at a corresponding degree of freedom in the first body coordinate system and a second offset compensation of the at least one motion execution unit at the rear end at a corresponding degree of freedom in the second body coordinate system, and drive, based on a preset motion priority and each of the first offset compensations and the second offset compensation, the at least one pair of forceps and a corresponding motion execution unit to automatically implant the electrode.

2. The implantation system according to claim 1, wherein the input unit comprises an operating handle, a user inputs a first driving instruction by the operating handle, and the first driving instruction comprises a motion direction and a motion amount of the at least one pair of forceps in the world coordinate system; the control unit converts the first driving instruction into a second driving instruction; the second driving instruction comprises the first offset compensations of the at least one pair of forceps and the at least one motion execution unit at the front end at the corresponding degree of freedom in the first body coordinate system; the second driving instruction further comprises the second offset compensation of the at least one motion execution unit at the rear end at the corresponding degree of freedom in the second body coordinate system; and based on the second driving instruction and the preset motion priority, the at least one pair of forceps and the corresponding motion execution unit are driven to implement a manual implantation of the electrode.

3. The implantation system according to claim 2, wherein the front end comprises a first rotary motion execution unit comprising a first driving motor, a first holder, and at least one winding arm;
the first holder is fixedly connected to the first driving motor; the first holder comprises two extension arms disposed opposite to each other, and the electrode implantation device is located between the two extension arms and is hinged to the two extension arms;
the at least one winding arm has a first end hinged to the electrode implantation device and a second end coupled to a first output shaft of the first driving motor; and the second end of the winding arm is driven by the first driving motor to rotate about the first output shaft, such that the at least one pair of forceps rotate about hinge points of the two extension arms and the electrode implantation device.

4. The implantation system according to claim 3, wherein the front end further comprises a second rotary motion execution unit comprising a second driving motor; the first driving motor is coupled to a second output shaft of the second driving motor, and the second output shaft is perpendicular to the first output shaft; and the first rotary motion execution unit and the electrode implantation device are driven by the second driving motor to rotate about a central axis of the second output shaft in an integrated manner.

5. The implantation system according to claim 4, wherein the rear end comprises a first rectilinear motion execution unit comprising a third driving motor, a first gear, a first rectilinear motion mechanism, and a first guide mechanism;
a first end of the first rectilinear motion mechanism is fixedly connected to the second rotary motion execution unit; the first guide mechanism is sleeved on the first rectilinear motion mechanism and is slidably connected to the first rectilinear motion mechanism, and the first rectilinear motion mechanism is guided by the first guide mechanism to move linearly along a Y-axis direction parallel to a length direction of the second output shaft;
a first gear and guide groove structure extending along the Y-axis direction is formed at a bottom of the first guide mechanism; the third driving motor is fixedly connected to the first rectilinear motion mechanism; the first gear is coupled to an output shaft of the third driving motor and is engaged with the first gear and guide groove structure; and the first gear is driven to rotate, such that the first rectilinear motion mechanism drives the second rotary motion execution unit, the first rotary motion execution unit, and the electrode implantation device to move linearly along the Y-axis direction in an integrated manner.

6. The implantation system according to claim 5, wherein the rear end further comprises a second rectilinear motion execution unit comprising a second rectilinear motion mechanism, a connecting plate, a fourth driving motor, a fourth gear, and a second guide mechanism;
the connecting plate is fixedly disposed at a top of the first guide mechanism and does not interfere with the first rectilinear motion mechanism; the second rectilinear motion mechanism is fixedly disposed on a top surface of the connecting plate; the second guide mechanism is sleeved on the second rectilinear motion mechanism and is slidably connected to the second rectilinear motion mechanism, and the second rectilinear motion mechanism is guided by the second guide mechanism to move linearly along an X-axis direction perpendicular to the Y-axis direction;
a second gear and guide groove structure extending along a length direction of the X axis is formed on a side of the connecting plate facing away from the electrode implantation device;
the fourth driving motor is fixedly connected to the second guide mechanism; the fourth gear is coupled to an output shaft of the fourth driving motor and is engaged with the second gear and guide groove structure; and the fourth gear is driven to rotate, such that the second rectilinear motion mechanism drives the first rectilinear motion execution unit, the second rotary motion execution unit, the first rotary motion execution unit, and the electrode implantation device to move linearly along the X-axis direction in an integrated manner.

7. The implantation system according to claim 6, wherein the rear end further comprises a third rotary motion execution unit comprising a first arc-shaped seat, a second arc-shaped seat, a third rotary motion mechanism, a fifth driving motor, and a fifth gear;
the first arc-shaped seat and the second arc-shaped seat are concentric and have a same radius; the first arc-shaped seat is fixedly disposed at a top of the second arc-shaped seat; an inner arc surface of the first arc-shaped seat faces the electrode implantation device; an outer arc surface of the first arc-shaped seat is provided with an arc-shaped gear and guide groove structure extending along a circumferential direction of the first arc-shaped seat; an inner arc surface and an outer arc surface of the second arc-shaped seat are provided with protrusions protruding outwards, and the protrusions extend along a circumferential direction of the second arc-shaped seat;
the third rotary motion mechanism is disposed below the second arc-shaped seat, and a top surface of the third rotary motion mechanism is fixedly provided with a plurality of upright columns located on an inner side and an outer side of the second arc-shaped seat respectively; outer walls of the plurality of upright columns are sunken inwards to form recesses, and the protrusions are partially embedded into the recesses; the protrusions are matched with the recesses, such that the second arc-shaped seat guides the third rotary motion mechanism to rotate about a Z axis being a central axis of the second arc-shaped seat that is perpendicular to the X axis and a Y axis;
the third rotary motion mechanism is further fixedly connected to the second guide mechanism and the fifth driving motor; the fifth gear is coupled to an output shaft of the fifth driving motor and is engaged with the arc-shaped gear and guide groove structure; and the fifth gear is driven to rotate, such that the third rotary motion mechanism drives the second rectilinear motion execution unit, the first rectilinear motion execution unit, the second rotary motion execution unit, the first rotary motion execution unit, and the electrode implantation device to rotate about the Z axis in an integrated manner.

8. The implantation system according to claim 7, wherein the rear end further comprises a third rectilinear motion execution unit comprising a third guide mechanism, a sixth driving motor, a sixth gear, and a third rectilinear motion mechanism;
the third guide mechanism is fixedly connected to an external mechanism and the sixth driving motor; the third guide mechanism is sleeved on the third rectilinear motion mechanism and is slidably connected to the third rectilinear motion mechanism; the third rectilinear motion mechanism is guided by the third guide mechanism to move linearly along a direction parallel to the Z axis;
the third rectilinear motion mechanism is fixedly connected to the second arc-shaped seat; the third rectilinear motion mechanism further comprises a third gear and guide groove structure extending along a Z-axis direction;
the sixth gear is coupled to an output shaft of the sixth driving motor and is engaged with the third gear and guide groove structure; and the sixth gear is driven by the sixth driving motor to rotate, such that the third rectilinear motion mechanism drives the third rotary motion execution unit, the second rectilinear motion execution unit, the first rectilinear motion execution unit, the second rotary motion execution unit, the first rotary motion execution unit, and the electrode implantation device to move linearly along the Z-axis direction in an integrated manner.

9. The implantation system according to claim 8, wherein the electrode implantation device further comprises at least one first implantation motor, and one of the at least one first implantation motors is corresponding to a pair of forceps of the at least one pair of forceps; the at least one pair of forceps are driven by the at least one first implantation motor to move linearly along a direction perpendicular to the first output shaft;
the first rotary motion execution unit and the second rotary motion execution unit have a same motion priority;
the first rectilinear motion execution unit and the second rectilinear motion execution unit have a same motion priority; and
motion priorities of the first implantation motor, the first rotary motion execution unit, the first rectilinear motion execution unit, the third rotary motion execution unit, and the third rectilinear motion execution unit decrease progressively in sequence.

10. A method of implanting a cochlear implant microelectrode, comprising:
operating in at least one of an automatic implantation mode, or in a manual implantation mode;
in response to operating in the automatic implantation mode,
inputting a surgical trajectory, wherein the surgical trajectory comprises multiple pieces of target information of a pair of forceps;
calculating, based on current target information and next target information of the pair of forceps, first offset compensations of the pair of forceps and a motion execution unit at a front end at a corresponding degree of freedom in a first body coordinate system and a second offset compensation of a motion execution unit at a rear end at a corresponding degree of freedom in a second body coordinate system; and
driving, based on a preset motion priority and each of the first offset compensations and the second offset compensation, the pair of forceps and a corresponding motion execution unit to automatically implant an electrode; and
in response to operating in the manual implantation mode,
inputting a first driving instruction by an operating handle, wherein the first driving instruction comprises a motion direction and a motion amount of the pair of forceps in a world coordinate system;
converting, by a control unit, the first driving instruction into a second driving instruction, wherein the second driving instruction comprises the first offset compensations of the pair of forceps and the motion execution unit at the front end at the corresponding degree of freedom in the first body coordinate system, and the second driving instruction further comprises the second offset compensation of the motion execution unit at the rear end at the corresponding degree of freedom in the second body coordinate system; and
driving, based on the second driving instruction and the preset motion priority, the pair of forceps and the corresponding motion execution unit to implement a manual implantation of the electrode.
